(19) United States
(12) Patent Application Publication
(10) Pub. No.: US 2025/0241862 A1
(43) Pub. Date: Jul. 31, 2025

(54) EXOSOMAL COMPOSITIONS INCLUDING ANESTHETICS

(71) Applicant: THE BOARD OF TRUSTEES OF THE UNIVERSITY OF ALABAMA, Tuscaloosa, AL (US)

(72) Inventors: Chao Zhao, Tuscaloosa, AL (US); Yuhao Cai, Tuscaloosa, AL (US)

(21) Appl. No.: 18/959,803

(22) Filed: Nov. 26, 2024

Related U.S. Application Data
(60) Provisional application No. 63/627,124, filed on Jan. 31, 2024.

(51) Int. Cl. A61K 9/50; A61K 31/445; A61K 31/529; A61P 23/02

(52) U.S. Cl. CPC A61K 9/5068; A61K 31/445; A61K 31/529; A61P 23/02

(57) ABSTRACT

Disclosed here are compositions including exosomes loaded with therapeutic agents, for example one or more anesthetic agents. The composition may be used to treated nerve pain, including peripheral nerve pain.

### CROSS-REFERENCE TO RELATED APPLICATION

This application claims the benefit of U.S. Provisional Application 63/627,124, filed Jan. 31, 2024, the contents of which are hereby incorporated in its entirety.

### STATEMENT OF GOVERNMENT SUPPORT

This invention was made with government support under grant/contract number R01GM144388 and R61NS123196 awarded by the National Institutes of Health. The government has certain rights in the invention.

### FIELD OF THE INVENTION

The disclosure relates to exosomal compositions including one or more therapeutic agents.

### BACKGROUND

Injection of clinically used anesthetics around peripheral nerves can effectively inhibit axonal signal transduction and relieve pain. However, less than 1% of the perineurally injected local anesthetics are able to cross the restrictive peripheral nerve barriers (PNBs), particularly the perineurium (an endothelial-like structure that encloses bundles of nerve fibers, which is a rate-limiting barrier for local anesthetic to cross before acting on axons), and ultimately act on peripheral nerve axons. This limitation necessitates the use of higher doses of local anesthetics, but the duration of a typical nerve block or infiltrations nerve block is still relatively short (2-3 hours), reflecting clearance of the molecule. Conversely, a significant portion of the injected anesthetics is absorbed by adjacent tissues or enters the systemic circulation, thereby increasing the risk of intrinsic muscle and nerve toxicity, as well as cardiovascular and neurologic systemic side effects.

The remains a need for improved compositions and methods for delivering anesthetics to specific tissues and locations in a body. There remains a need for improved compositions and methods for selectively delivering anesthetics to specific tissues with improved pharmacokinetics and reduced systemic exposures.

### BRIEF DESCRIPTION OF THE FIGURES

FIG. 1 depicts a schematic illustration demonstrating the mechanism of sciatic nerve block using exosomal bupivacaine. Exosomes penetrate the peripheral nerve barriers. Subsequently, bupivacaine is slowly released and diffuses throughout the nerve, and eventually acts on the axons.

FIG. 2 determination and analysis of the penetrative performance of exosomes through PNB. a. Representative confocal fluorescent microscopy images of the nerve section with injections of fluorescent-labeled exosomes (Labelled EXO), scale bar=100 um. b. Zoom-in illustration of confocal fluorescent microscopy images for labeled exosomes(a), scale bar=100 um. c. Representative confocal fluorescent microscopy images of the nerve section with injections of the free ExoGlow™-Protein dye, scale bar=100 um. d. Zoom-in illustration of confocal fluorescent microscopy images for free ExoGlow™-Green dye(c), scale bar=100 um. e. Normalized mean fluorescent intensity analysis based on the confocal fluorescent microscopy images. Normalized distance=(distance[ai] from the border)/(average diameter of the fiber[d]); normalized mean fluorescent intensity=(mean fluorescent intensity[Intᵢ] at distance[a;])/(mean fluorescent intensity at the border[outermost ring, Int₁]), n=4.

FIG. 3 depicts preparation and in vitro characterization of exosomal bupivacaine (BUP@EXO). a. Schematic diagram of the preparation procedure of exosomal bupivacaine. b. DLS measurements for hydrodynamic diameter of native exosomes (native EXO) and exosomal bupivacaine (BUP@EXO). c. A representative TEM image of native exosomes and the corresponding size distribution. d. A representative TEM image of exosomal bupivacaine and the corresponding size distribution. e. In vitro release of bupivacaine from exosomal bupivacaine (BUP@EXO), free bupivacaine solution with 0.5% (w/v) concentration (0.5% BUP) and saturated concentration (Saturated BUP) in PBS at 37° C. n=3. f. Immunoblot analysis of exosome markers (CD63, CD81, EpCAM, ANXA5, TSG101, ALIX, ICAM, FLOT1, GM130) of the native exosomes and BUP@EXO formulations.

FIG. 4 Storage stability assessment for exosomal bupivacaine (BUP@EXO). a. Evaluation of loading stability of BUP@EXO at −20° C., 4° C. and 25° C., n=4. b. Dynamic mean diameter measured by DLS of BUP@EXO particles stored at −20° C., 4° C. and 25° C., n=4. c. Surface zeta potential of exosomal bupivacaine particles stored at −20° C., 4° C. and 25° C., n=4; *, p<0.05 vs value at Time=0.

FIG. 5 depicts neurobehavioral assessment of rats that received a single sciatic nerve injection of exosomal bupivacaine (BUP@EXO) and free bupivacaine (BUP). Effects on BUP dosage on frequency of successful sensory nerve blockade (a), duration of sensory nerve blockade (b), and duration of motor nerve blockade (c), n=8. d. Representative profiles of thermal latency and bearing weight of rats measured at specific intervals post injections on both ipsilateral and contralateral paw legs. *, p<0.25, ****, p<0.0001.

FIG. 6 depicts Local tissue and systemic toxicity evaluation of exosomal bupivacaine (BUP@EXO) and free bupivacaine (BUP). a. Representative H&E-stained sections of the muscle at the injection site. M: Muscle; N: Nerve region; Inf: inflammation; Mtox: myotoxicity. b. Representative toluidine blue-stained sections of the sciatic nerve from the injection site. c. CK-total activity, 48 h post injection, n=4. d. CK-MB activity, 48 h post injection, n=4. **, p<0.01, **** p<0.0001.

FIG. 7 depicts myotoxicity and inflammation scores (48 hours) for bupivacaine-loaded exosomes compared with non-exosomal bupivacaine and blank exosomes.

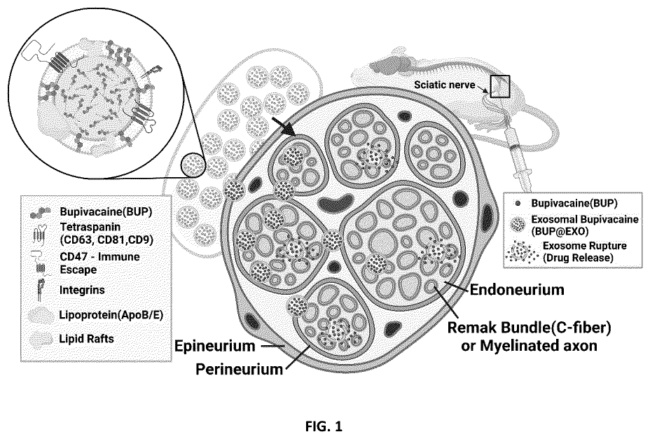

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The therapeutic agents disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Unless an agent is specifically designated a free base, free acid, or a specific salt (e.g., hydrochloride), reference to that agent is intended to embrace both salt and non-salt forms of the agent. Unless specified explicitly to the contrary, reference to particular dosage or amount of a particular agent is with reference to the free base. By way of example, 10 mg of “bupivacaine” (free base MW=288.43 g/mol) also describes 11.26 mg of bupivacaine hydrochloride (MW=324.9 g/mol).

Disclosed herein are compositions including an anesthetic encapsulated in an exosome.

### Anesthetics

In some implementations the anesthetic can be a local anesthetic, a sodium channel blocker, a peptide neurotoxin, a TRPV1 antagonist, a TRPV1 agonist, a COX inhibitor (either COX-1 or COX-2), NMDA antagonist, a GABA agonist, a barbiturate, or a combination thereof.

In some implementations, the local anesthetic can be acetamidoeugenol, alfadolone acetate, alfaxalone, amucaine, amolanone, amylocaine, articaine, benoxinate, benzocaine, betoxycaine, biphenamine, bupivacaine, burethamine, butacaine, butaben, butanilicaine, buthalital, butoxycaine, carticaine, centbucridine, 2-chloroprocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutyl p-aminobenzoate, ketamine, leucinocaine mesylate, levoxadrol, lidocaine, lignocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, sameridine, tetracaine, thialbarbital, thimylal, thiobutabarbital, thiopental, tolycaine, trimecaine, and zolamine, a pharmaceutically acceptable salt thereof, and combinations thereof.

In some implementations, the composition can include dibucaine, lidocaine, bupivacaine, benzocaine, tetracaine, prilocaine, pharmaceutically acceptable salts thereof, and combinations thereof.

In some implementations, the composition can include bupivacaine hydrochloride.

In certain implementations, the composition can include a TRPV1 antagonist, a TRPV1 agonist, or a combination thereof.

In some implementations the TPRV1 agonist can selected from N-vanillyl-alkanedienamides, N-vanillyl-alkanedienyls, N-vanillyl-cis-monounsaturated alkenamides, gingerol, shogaol, zingerone, eugenol, N-arachidonoyldopamine, olvanil, palvanil, resiniferatoxin, capsaicin, dihydrocapsaicin, norhydrocapsaicin, nordihydrocapsaicin, homocapsaicin, and homodihydrocapsaicin, pharmaceutically acceptable salts thereof and combinations thereof.

In some implementations the TPRV1 antagonist can be selected from capsazepine, ruthenium red, pharmaceutically acceptable salts thereof, and combinations thereof.

In certain implementations, the composition can include a site 1 calcium channel blocker. The site 1 calcium channel blocker can be selected from tetrodotoxin, batrachotoxin, saxitoxin, gonyautoxin, neosaxitoxin, decarbamoylsaxitoxin, pharmaceutically acceptable salts thereof, and combinations thereof.

In certain implementations, the composition can include a peptide neurotoxin. The peptide neurotoxin can be selected from agatoxin, conotoxin, calciseptine, calcicludine, kurtoxin, cysteine-rich secretory proteins (CRISPs), glycerotoxin, phonetoxin, filistatoxin, SNX-482, protoxins, grammotoxin, pharmaceutically acceptable salts thereof, and combinations thereof.

In some implementations the composition can include a protein neurotoxin, for example botulinum neuortoxin.

When the composition includes a local anesthetic, the local anesthetic can be present in an amount (in free base equivalents) from 0.1-100 mg, 0.1-50 mg, from 0.1-25 mg, from 0.1-10 mg, from 0.1-5 mg, from 0.1-2.5 mg, from 0.1-1 mg, from 0.5-5 mg, from 1-5 mg, from 2.5-5 mg, from 5-10 mg, from 10-25 mg, or from 25-50 mg.

In some implementations the composition includes bupivacaine, or a pharmaceutically acceptable salt thereof, in an amount (in free base equivalents) from 0.1-50 mg, from 0.1-25 mg, from 0.1-10 mg, from 0.1-5 mg, from 0.1-2.5 mg, from 0.1-1 mg, from 0.5-5 mg, from 1-5 mg, from 2.5-5 mg, from 5-10 mg, from 10-25 mg, or from 25-50 mg. In further implementations the bupivacaine salt is bupivacaine hydrochloride.

In some implementations the composition includes a site 1 calcium channel blocker in an amount (in free base equivalents) from 0.1-50 μg, from 0.1-25 μg, from 0.1-10 μg, from 0.1-5 μg, from 0.1-2.5 μg, from 0.1-1 μg, from 0.5-5 μg, from 1-5 μg, from 2.5-5 μg, from 5-10 μg, from 10-25 μg, or from 25-50 μg. In certain implementations the site 1 calcium channel blocker is tetradotoxin.

### Exosomes

In certain implementations, the composition can include an exosome derived from an animal cell, a plant cell, a fungal cell, or a bacterial cell. For example, in some embodiments, the exosome is derived from a mammalian cell, and in certain embodiments, from a mammalian stem cell.

In certain implementations, the composition includes an exosome derived from a mammalian embryonic stem cell or induced pluripotent stem cell. In some implementations, the exosome is derived from a human embryonic kidney cell, for example human embryonic kidney 293 cell.

In some embodiments, the exosome has an average particle diameter (measured by dynamic light scattering) from 50-1000 nm, from 100-1000 nm, from 250-1000 nm, from 500-1000 nm, from 50-500 nm, from 100-500 nm, from 150-500 nm, from 250-500 nm, from 50-250 nm, from 100-250 nm, from 150-250 nm, from 200-250 nm, from 100-150 nm, or from 150-200 nm.

### Compositions

The compositions disclosed herein can include one or more pharmaceutically acceptable excipients. The compositions may be formulated as aqueous formulations for injection, using sterile water or suitable buffer solution. In certain implementations the composition can be formulated in buffer comprising PBS, HEPES, HEPPS, HEPBS, PIPES, TAPS, Tris, TAPSO, MOPS, MES, cacodylate, TES, tricine, or a combination thereof.

The composition can further include a preservative, for example benzyl alcohol, benzalkonium chloride, butyl paraben, chlorobutanol, meta cresol, chlorocresol, methyl paraben, phenyl ethyl alcohol, propyl paraben, phenol, benzonic acid, sorbic acid, sodium benzonate, bronidol, propylene glycol, or a combination thereof.

The composition can further include a surfactant, for example tocopheryl polyethylene glycol succinate, polyethylene glycol, Triton X-100, Tween 80, poloxamers, polysorbates, polidocanol, pentaethylene glycol monododecyl ether, lauryl glucoside, maltoside, monolaurin, mycosubtilin, narrow-range ethoxylate, nonidet P-40, nonoxynols, NP-40, octaethylene glycol monododecyl ether, N-octyl beta-D-thioglucopyranoside, octyl glucoside, oleyl alcohol, alkyl polyglycoside, cetomacrogol, cetostearyl clcohol, cetyl alcohol, cocamides, decyl glucoside, decyl polyglucose, glycerol monostearate, IGEPAL, isoceteth-20, or a combination thereof.

The composition can further include an antioxidant, for example vitamin A, vitamin A derivative, vitamin C, vitamin C derivatives, vitamin E, vitamin E derivatives, polyphenols, beta-carotene, lutein, lycopene, selenium, or a combination thereof.

The composition can include one or more oils. Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In certain implementations the composition is an injectable composition or a topically applied composition. Injectable compositions can be aqeuous compositions, optionally having buffers and excipients as described above. Topically applied compositions include lotions, ointments, sprays, creams and the like, which may be obtained by combining the exosomes with a pharmaceutically acceptable vehicle, for example a thickening agent. Topically applied compositions may include the exosomes in concentrations from 1-25 wt. %, from 1-10 wt. %, from 1-5 wt. %, from 5-10 wt. %, or from 10-25 wt. %. In certain implementations, the composition is a lyophilized composition, optionally in combination with one or more buffers and excipients as described above. Lyophilized compositions can be reconstituted in a suitable aqeuous vehicle for injection.

In certain implementations, the composition is provided in a topically applied patch form, including the compositions disclosed herein and a suitable backing layer.

The exosomes disclosed herein exhibit cold storage stability. For example, in certain implementations, an aqeuous composition including anesthetic-loaded exosomes at 1 mg anesthetic/mL will lose 10% or less of the anesthetic over a period of two weeks when stored at 4 C and will lose 20% or more of the anesthetic over a period of 48 hours when stored at 23° C.

### Methods of Manufacture

The compositions disclosed herein can be prepared by contacting an exosome with a solution including an anesthetic and optionally one or more additional excipients as disclosed herein. The solution can have a temperature from 23-40° C., from 23-37° C., from 30-37° C., or from 33-37° C. Exemplary solutions include buffered aqeuous solutions, for example 1×PBS. In certain implementations, the solution can also include a water-soluble organic solvent at a concentration that is from 0.1-5% (v/v), from 0.5-5% (w/v/), from 1-5% (v/v), from 2.5-5% (w/v/), from 0.1-1% (v/v), from 0.1-0.5 w % (v/v), from 0.25-0.75% (v/v) from 0.5-1% (v/v). In certain implementations the water-soluble organic solvent is DMSO.

In certain implementations, the solution can include the anesthetic (for example a local anesthetic) at a concentration from 1-250 mg/ml, from 1-100 mg/ml, from 1-75 mg/ml, from 1-50 mg/ml, from 1-25 mg/ml, from 1-10 mg/ml, from 5-15 mg/ml, from 10-20 mg/ml, from 10-50 mg/ml, from 25-75 mg/ml, or from 50-100 mg/ml. In other implementations, when the anesthetic is a site 1 calcium channel blocker, the solution can include the anesthetic at a concentration of 1-250 μg/ml, from 1-100 μg/ml, from 1-75 μg/ml, from 1-50 μg/ml, from 1-25 μg/ml, from 1-10 μg/ml, from 5-15 μg/ml, from 10-20 μg/ml, from 10-50 μg/ml, from 25-75 μg/ml, or from 50-100 μg/ml.

The contacting of the exosome with the solution including the anesthetic may be designated the loading phase. Following the loading phase, the loaded exosome may be subjected to one or more free-thaw cycles, wherein the loaded exosome is first subjected to a freeze period at a temperature from −120° C. to −50° C., followed by a thaw period at a temperature from 10-40° C. In some implementations, the freeze period can be at a temperature from −90° C. to −70° C., preferably about −80° C., and the thaw period can be at a temperature from 23-40° C., preferably about 37° C.

In certain implementations, the loaded exosome can be subjected to more than 1 freeze-thaw cycle. In certain implementations, the loaded exosome is subjected to 2-6 (i.e., 2, 3, 4, 5, or 6) freeze-thaw cycles, to 2-4 freeze-thaw cycles, to 3-5 freeze-thaw cycles, or to 4-6 freeze-thaw cycles. In some implementations, the loaded exosome is subjected to 3 freeze-thaw cycles.

In some implementations, the freeze period is for a duration from 2-30 minutes, from 2-20 minutes, from 2-15 minutes, from 2-10 minutes, from 2-5 minutes, from 4-10 minutes, from 8-15 minutes, or from 10-30 minutes.

In some implementations, the thaw period is for a duration from 2-30 minutes, from 2-20 minutes, from 2-15 minutes, from 2-10 minutes, from 2-5 minutes, from 4-10 minutes, from 8-15 minutes, or from 10-30 minutes.

Following freeze/thaw cycling, the loaded exosomes may be purified by washing with water or buffer solution (for example 1×PBS) and isolated via centrifugation or filtering. The washing step may be repeated as necessary.

In some implementations, the exosomes are loaded with a therapeutic agent using an osmotic shock process. The exosomes may first be dialyzed against pure water for a first period, then dialyzed against a solution including the therapeutic agent for a second period, and then dialyzed against a hypertonic buffer (for example 1×PBS) to reach a final osmolality of about 300 mOsmol/kg.

### Methods of Use

The compositions disclosed herein may be used for producing anesthesia in a patient in need thereof, by administering to the patient a composition including an anesthetic-loaded exosome.

In certain implementations, the composition is administered by injection, for example to a location adjacent to a nerve. In some implementations, the composition is administered by injection to a location adjacent to a peripheral nerve. In certain implementations, the composition is administered by injection to a location adjacent to the celiac plexus nerve, the epidural nerve, the genicular nerve, the intercostal nerve, the lumbar sympathetic nerve, the occipital nerve, the pudendal nerve, the stellate ganglion nerve, or the trigeminal nerve.

### EXAMPLES

The following examples are for the purpose of illustration of the invention only and are not intended to limit the scope of the present invention in any manner whatsoever.

### Example 1—Synthesis and Characterization of Anesthetic-Loaded Exosomes

Exosomes derived from HEK 293 cells (1 mcg mcL⁻¹, 50 mcg per vial, equivalent to >106 vesicles), and the ExoGlow™-Protein EV Labeling Kit were purchased from System Biosciences, LLC (Palo Alto, CA, USA). Bupivacaine Hydrochloride (“BUP”) was purchased from Tokyo Chemical Industry Co., Ltd. (TCI, Tokyo, Japan).

The exosomes were fluorescently labeled utilizing the ExoGlow™-Protein EV Labeling Kit. In brief, exosomes equivalent to 200-500 μg of protein were resuspended in 200 μL of 1× PBS. Subsequently, 1 μL of the 500× labeling dye was added to the exosome preparation. This mixture was incubated at 37° C. with gentle agitation at 350 rpm for a duration of 20 minutes. Following incubation, 167 μL of ExoQuick-TC solution was introduced to the mixture and further incubated at 4° C. for a period ranging from 2 hours to overnight. The mixture was then centrifuged at 10,000 rpm for 10 minutes. The supernatant was carefully aspirated from the tube's corner, ensuring the exosome pellet remained undisturbed. The pellet was then resuspended in PBS, ready for downstream applications.

A BUP solution with a concentration of 10 mg/mL in 1×PBS with an addition of 0.5% DMSO was combined with an exosome solution at 1 mg/mL in 1×PBS. This mixture was then incubated at 37° C. with gentle agitation at 350 rpm, with an overnight duration (18 hours). Subsequently, the solution underwent three consecutive freeze-thaw cycles, alternating between −80° C. and 37° C., 5 mins for each temperature. Subsequently, the mixture underwent a washing process using 10 mL of 1×PBS in centrifugal concentrators, repeated thrice at 3,500 rpm for 30 min. The final retentate in the upper chamber, approximately 200 μL, was harvested as the product. This was then diluted to the desired concentration for subsequent experiments. The washing solution (filtrate) in the lower chamber was reserved for encapsulation efficiency (EE) assessment. The concentration of BUP was determined by measuring the absorbance at 272 nm and comparing it to a pre-established standard curve.

Transmission electron microscopy (TEM) was employed to visualize both empty exosomes and BUP@EXO. Initially, 10 μL of both empty and loaded exosomes were mixed with an equal volume of 4% paraformaldehyde aqueous solution, followed by a 30-minute fixation at 4° C. Subsequently, the fixed exosomes were carefully placed onto carbon-formvar-coated copper grids (300 mesh) and allowed to dry for 20 minutes. Excess liquid was gently removed using filter paper. The grids were then subjected to three washes with deionized water, each lasting 1 minute. For contrast enhancement, the grids were immersed in a 10 μL drop of 2% uranyl acetate and shielded from light for 5 minutes. After this, the grids underwent another three washes with deionized water, each lasting 2 minutes. The prepared samples were then examined under a transmission electron microscope (Hitachi, Tokyo, Japan). The hydrodynamic size in PBS was measured using a Malvern (Malvern, UK) Zetasizer Nano series dynamic light scattering instrument.

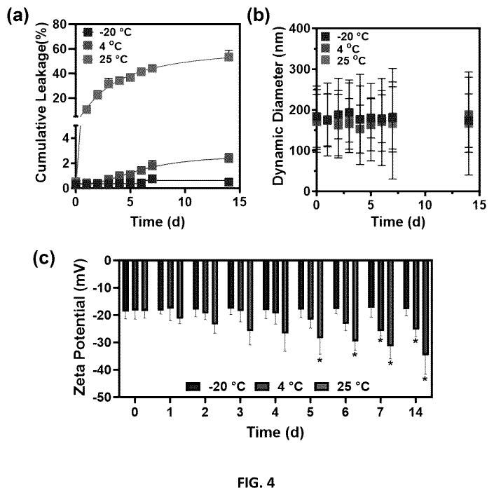

Intact native exosomes and BUP-loaded exosomal formulations(BUP@EXO) were characterized by dynamic light scattering (DLS) and transmission electron microscopy (TEM) imaging. The hydrodynamic diameter (z-average) and polydispersity index (PDI) values obtained from DLS revealed that native exosomes exhibited a diameter of 121.1±30.20 nm with a low polydispersity (PDI=0.249) (FIG. 3b). In comparison, BUP@EXO displayed particles with a diameter of 184.6±67.41 nm and a low polydispersity (PDI=0.262), indicating the BUP loading slightly increased the hydrodynamic size of the exosomes. The zeta potential of native exosomes was measured at −21.3±2.7 mV, while that of the BUP@EXO showed an increase to −18.6±3.1 mV (FIG. 3c). The increase in zeta potential can be attributed to the neutral nature of Bup, and the BUP loading reduces the surface charge density of the exosomes. TEM imaging was then used to determine whether the morphology of the exosomes was maintained after the BUP encapsulation. As illustrated in FIGS. 3d and 3e, BUP@EXO maintained spherical shapes similar to those of native exosomes, indicating the preservation of structural integrity. Diameter analysis via TEM corroborated the DLS results, with native exosomes exhibiting a diameter of 100.8±23.70 nm, compared to the BUP@EXO which measured at 164.2±53.31 nm.

To assess the release kinetics of BUP from the BUP@EXO, a dialysis method was employed. Specifically, 1 mL of BUP@EXO, containing 5 mg BUP, was introduced into a Spectra/Por Float-A-Lyzer G2 dialysis bag with a molecular weight cutoff ranging from 8-10 kD (Spectrum Laboratories, Ranchodominguez, CA). This bag was then immersed in 20 mL of phosphate-buffered saline (PBS) with a pH of 7.4. The setup was maintained at 37° C. and subjected to continuous 200 rpm shaking. At specified time points, the dialysis bag was relocated to fresh 1×PBS to ensure a consistent concentration gradient. The concentration of BUP in the dialysate was ascertained by measuring its absorbance at 272 nm and referencing it against a pre-established standard curve. We collected the washout solutions (filtrate) in the lower chambers of centrifugal filters. Subsequently, these solutions underwent UV-Vis spectroscopy to determine the BUP concentration. The encapsulation efficiency is thereby calculated by

$$EE = \frac{m ( total BUP ) - m ( Washed out BUP )}{m ( total BUP )} .$$

Our results indicated a consistent EE for BUP@EXO, maintaining between 76.64% and 82.33%, which translates to approximately 15.33 to 16.45 μg BUP per μg of protein-equivalent exosome. The observed consistency (p=0.2922, single factor ANOVA) in EE across different incubation batches highlights the robustness of our methodological approach. This consistency closely aligns with previously published results [34, 36, 37]. Notably, we observed a slight elevation in EE, which could be attributed to the gentler centrifugal filtration steps employed in our process compared with ultracentrifuge or ultrafiltration, which are the conventional methods applied in previous research.

We conducted in-depth in vitro drug release kinetic analysis at 37° C. for our BUP@EXO formulations. 1 mL of BUP@EXO in 1×PBS, containing 5 mg BUP, was introduced into a dialysis bag with a molecular weight cutoff ranging from 8-10 kD. The dialysis bag was then immersed in 20 mL of 1×PBS with a pH of 7.4. The setup was maintained at 37° C. and subjected to continuous shaking of 200 rpm. The results revealed that the BUP@EXO substantially prolonged the release duration of BUP, exhibiting a statistically significant difference in comparison to free BUP at both 0.5% (w/v) and saturated concentrations (notably, P<0.001 at the 5-hour mark; FIG. 3e). Specifically, while the free BUP molecules at both concentrations approached a near-complete cumulative release within an initial 5-hour period, the BUP@EXO demonstrated a markedly slower release rate, remaining below 10%. Remarkably, the time frame required for BUP@EXO to achieve approximately 100% release extended to about 672 hours (FIG. 3e). These findings, indicative of a sustained release mechanism spanning several days, bolster the proposition that BUP@EXO holds significant promise for facilitating a long-lasting local anesthetic effect.

To assess the stability of BUP@EXO, we stored our BUP@EXO samples at both 4° C. and 25° C. At specified intervals (24 h, 48 h, 72 h, 96 h, 120 h, 144 h, 168 h, and 14 days), 1 mL of the BUP@EXO containing 1 mg BUP was centrifuged with a centrifugal filter (3,000 MW Millipore) at 3,500×g, for 30 min, at 4° C. The exosomes were retained in the upper chamber; 100-150 μL of the filtrate was recovered from the lower chamber. The BUP concentration in this wash solution was determined by measuring its absorbance at 272 nm and comparing it to a previously established standard curve.

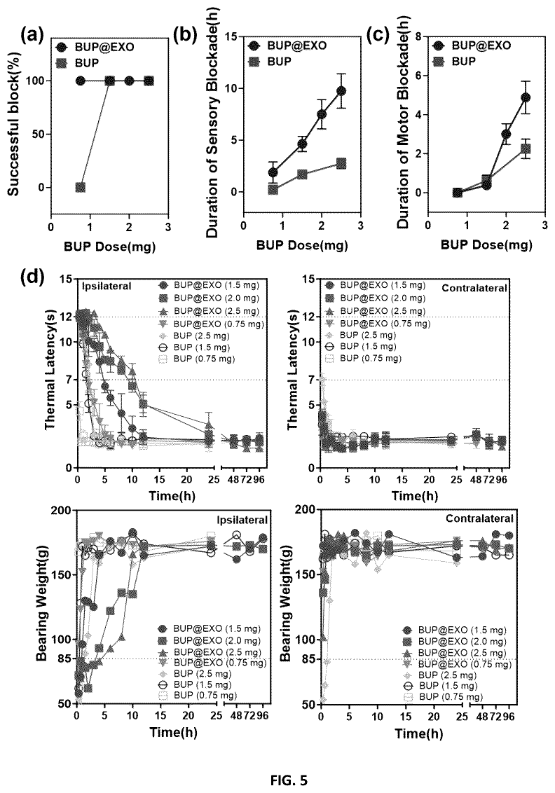

We assessed the stability of BUP@EXO formulations by measuring the leakage of bupivacaine from the exosomes over time at three different temperatures: −20° C., 4° C. and 25° C., representing frozen, refrigerated, and ambient conditions, respectively. It is crucial to highlight that we employed centrifugal filtration to evaluate bupivacaine leakage from the exosomes, deviating from the dialysis method utilized for release kinetic analysis. Briefly, 1 mL of BUP@EXO formulation containing 1 mg BUP equivalents were stored at −20° C., 4° C. and 25° C. At specific intervals, i.e., every 24 hours, 400 μL of the BUP@EXO was centrifuged with a centrifugal filter (with a molecular weight cut off=3 kD) at 3,500×g, for 30 min, at 4° C. The exosomes were retained in the upper chamber; 100-150 μL of the filtrate was recovered from the lower chamber[39]. The BUP concentration in the filtrate was determined by UV-Vis. Results showed that the BUP@EXO formulations exhibited temperature-dependent storage stability. At −20° C., over a 14-day period, the formulations exhibited robust stability with less than 1% leakage of encapsulated BUP (FIG. 4a). At 4° C., minimal drug leakage was observed, registering at less than 3% throughout the 14-day period, which is comparable to that at −20° C. (FIG. 4a). This suggests that the BUP encapsulation in exosomes can be maintained under both frozen and refrigerated conditions. In contrast, at 25° C., the BUP@EXO formulation showed marked instability, with BUP leakage exceeding 20% within the first 48 hours and reaching an average of 53.32% after 14 days (FIG. 4a).

In addition to monitoring BUP leakage, we assessed other critical attributes for exosome stability, including hydrodynamic diameter, zeta potential, and the presence of exosomal proteins. At −20° C., the mean dynamic diameter of BUP@EXO consistently remained within the 95% confidence interval (CI) of its initial distribution (p>0.05, FIG. 4b), with a stable zeta potential of −17.98±2.3 mV (p>0.05, FIG. 4d)observed over two weeks. No precipitates were detected in the solutions during this period (FIG. 4c, FIG. S1). At 4° C., the mean dynamic diameter also stayed within the 95% CI (FIG. 4b), and the solutions remained clear, free of visible precipitates (FIG. 4c). However, a gradual decrease in zeta potential from −18.3±3.2 mV to −24.3±2.6 mV (p=0.0251, FIG. 4d) over 14 days suggested alterations in the exosomal surface components, which possibly attributes to the inactivation or degeneration of the surface proteins, and the structure change of the phospholipid bilayer (FIG. 4d). At 25° C., while the solutions retained transparency and the mean dynamic diameter stayed within the 95% CI (FIG. 4b, 4c), a notable reduction in zeta potential from −18.5±2.6 mV to −34.7±6.9 mV (p=0.0003, FIG. 4d) was observed after 14 days, falling below that of native exosomes. This change indicates a potential compromise in the integrity of the exosomes, such as the rupture of exosomes.

### Example 2—In Vivo Evaluation of Anesthetic-Loaded Exosomes

Adult male Sprague-Dawley rats (Charles River Laboratories, Wilmington, MA) weighing 250-350 g were housed in groups, in a 6 am-6 pm light-dark cycle. Animals were cared for in accordance with protocols approved by the Institutional Animal Care and Use Committee of the University of Alabama (Protocol ID: 19-11-2992), and the Guide for the Care and Use of Laboratory Animals of the US National Research Council.

Nerve block injections were meticulously executed utilizing a 23-gauge needle, with rats being anesthetized under a combination of isoflurane and oxygen. The experimental design involved administering 0.3 mL of BUP@EXO at varying doses, specifically containing equivalent BUP amounts of 0.75 mg, 1.5 mg, 2.0 mg, and 2.5 mg. Additionally, injections of free BUP were given at doses of 0.75 mg and 1.5 mg. A control group received an injection of 50 μg of empty exosomes. The injection technique involved positioning the needle posteromedially to the greater trochanter and directing it anteromedially. Upon making contact with the bone, the prepared solution was administered.

Adult male Sprague-Dawley rats weighing 250-350 g (4 in each group) were administered injections at the left sciatic nerve with 0.3 mL of 1×PBS solutions containing free BUP or BUP@EXO. Subsequently, the rats underwent neurobehavioral test to determine the duration of functional deficits (i.e., sensory and motor nerve blockade) in both hind paws. The duration of deficits on the injected side (left, ipsilateral) reflected the duration of nerve block. Any deficits observed on the uninjected side (right, contralateral) were indicative of the systemic BUP distribution.

The efficacy of the nerve block was assessed at specified intervals using established methodologies. Sequentially, the left and then the right hind paws of the rats were subjected to a 56° C. hot plate (model 39D Hot Plate Analgesia meter; IITC Inc., Woodland Hills, CA). The duration until the paw was withdrawn, termed thermal latency, was meticulously recorded. This data provides insights into both the intensity (thermal latency) and the duration of the block. Thermal latency serves as an indicator of analgesic potency. To prevent potential injury or the onset of hyperalgesia, if the animal did not retract its paw within a 12-second window, it was manually removed. For a systemic effect baseline, latency was also gauged in the non-injected leg. The duration of the thermal nociceptive block was determined as the time taken for the thermal latency to revert from an elevated value to a baseline of 7 seconds. In adult rats, this 7-second threshold represents the median between the maximum block and the typical thermal latency, which is roughly 2 seconds, with the upper limit being 12 seconds. Motor function was evaluated by suspending the animal over a balance and recording the maximum weight the animal could bear. The duration of motor blockade was determined as the period until weight-bearing capacity returned to half of its normal level from the point of maximal block. For each rat, the halfway point was calculated as [(highest weight borne by either leg)−(lowest weight borne by the blocked leg)]/2+the lowest weight borne by the blocked leg.

The group of rats receiving sciatic nerve injections of free BUP presented a dose-dependent nerve blockade. It was observed that a low dose of 0.75 mg BUP in PBS resulted in neither a detectable nerve block nor toxicity, as evidenced in FIGS. 5a, 5b, and 5c. Conversely, a higher dose of 1.5 mg BUP in PBS successfully induced nerve block at the injected sites in all animals (FIG. 5a). This dosage yielded a duration of sensory nerve blockade of 1.833±0.250 hours, accompanied by a motor blockade lasting 0.458±0.176 hours (FIG. 5b, 5c). Comparative analysis of contralateral thermal latency and pre-injection baseline data revealed no contralateral nerve blockade in animals administered up to 1.5 mg of free BUP (p=0.8025 for 0.75 mg BUP, p=0.4858 for 1.5 mg BUP). Upon escalating the dosage to 2.5 mg, rats exhibited a sensory nerve blockade lasting 2.75±0.43 hours and a motor blockade of 2.12±0.54 hours (FIG. 5b, 5c)), accompanied by an occurrence of drowsiness (3 out of 4 animals). Notably, the administration of 2.5 mg of free BUP induced a contralateral sensory nerve blockade (3 out of 4 animals, thermal latency ˜7s, p<0.0001, FIG. 5d), suggesting a detectable systemic distribution of BUP at this dosage.

The group of rats receiving sciatic nerve injections of BUP@EXO formulation showed higher potency of BUP@EXO in sciatic nerve blockade and reduced bupivacaine-related systemic side effects compared to pure bupivacaine. At a dosage where free BUP failed to induce any nerve blockade, we administered BUP@EXO containing 0.75 mg BUP to the left sciatic nerve. This exosomal delivery significantly enhanced the success rate of nerve blockade from 0 to 100% (FIG. 5a), resulting in a sensory blockade lasting 2.12±0.629 hours. Notably, this administration did not produce motor blockade (i.e., they were capable of bearing >50% of their weight on the injected limb, FIG. 5b, 5c, 5d), exhibiting a sensory-selective nerve blockade that aligns well with the clinical requirements for targeted pain management across a broader and more specific range of indications.[40-42]. It is worth noting that some animals, classified as not having received motor blocks, exhibited minimal degrees of motor impairment (i.e., slightly reduction in bearing weight at the injected side). This observed phenomenon of rendering ineffective BUP doses effective can be attributed to the exosomes' ability to facilitate BUP's passage across the PNB. In addition, the quantity of BUP that effectively penetrates and functions is only sufficient to induce sensory nerve blockade on unmyelinated neurons in Remak Bundles (C-fibers), without significantly impacting myelinated neurons. Upon administering BUP@EXO containing 1.5 mg of BUP in PBS, the rats demonstrated a sensory blockade lasting 4.556±0.726 hours, a duration notably double that of the same dose of free BUP. Additionally, a transient motor blockade of about 0.493±0.175 hours was observed, similar to that induced by the equivalent dose of free BUP. These findings further confirm that the exosomes effectively delivered BUP through the PNB, resulting in a prolonged and efficient sensory nerve blockade.

The administration of BUP@EXO containing 2.0 mg equivalent BUP induced a sensory nerve blockade for 7.556±1.333 hours, with a motor nerve blockade lasting 3.000±0.535 hours (FIG. 5b, 5c, 5d). When injected with BUP@EXO containing 2.5 mg equivalent BUP, a substantial sensory nerve blockade of 9.556±1.333 hours was observed, along with a motor blockade lasting 4.875±0.835 hours (FIG. 5b, 5c, 5d). The observed extension in nerve blockade duration emphatically highlights the efficacy of BUP@EXO in delivering prolonged local anesthesia. This efficiency is attributed to the dual mechanisms of sustained bupivacaine release from the exosomes and the effective penetration of these exosomes through the PNB. Notably, no contralateral nerve blockade was detected in the rats (p=0.6185 for 0.75 mg BUP@EXO, p=0.5839 for 1.5 mg BUP@EXO, p=0.3992 for 2.0 mg BUP@EXO, p=0.9026 for 2.5 mg BUP@EXO), indicating that while BUP@EXO effectively delivered more BUP into the PNB to act on axons, it concurrently resulted in reduced BUP entry into systemic circulation. This led to negligible systemic toxicity, highlighting the dual benefit of enhanced local nerve blockade efficacy, and minimized systemic side effects.

The group of rats receiving sciatic nerve injections of free BUP presented a dose-dependent nerve blockade. It was observed that a low dose of 0.75 mg BUP in PBS resulted in neither a detectable nerve block nor toxicity, as evidenced in FIGS. 5a, 5b, and 5c. Conversely, a higher dose of 1.5 mg BUP in PBS successfully induced nerve block at the injected sites in all animals (FIG. 5a). This dosage yielded a duration of sensory nerve blockade of 1.833±0.250 hours, accompanied by a motor blockade lasting 0.458±0.176 hours (FIG. 5b, 5c). Comparative analysis of contralateral thermal latency and pre-injection baseline data revealed no contralateral nerve blockade in animals administered up to 1.5 mg of free BUP (p=0.8025 for 0.75 mg BUP, p=0.4858 for 1.5 mg BUP). Upon escalating the dosage to 2.5 mg, rats exhibited a sensory nerve blockade lasting 2.75±0.43 hours and a motor blockade of 2.12±0.54 hours (FIG. 5b, 5c)), accompanied by an occurrence of drowsiness (3 out of 4 animals). Notably, the administration of 2.5 mg of free BUP induced a contralateral sensory nerve blockade (3 out of 4 animals, thermal latency ˜7s, p<0.0001, FIG. 5d), suggesting a detectable systemic distribution of BUP at this dosage.

Four hours after the injection, the rats were euthanized, and both the nerve and surrounding muscle tissues were harvested. These tissues were then embedded in OCT compound for cryoprotection and subsequently sectioned using a cryomicrotome. The tissue sections were visualized and imaged with a laser scanning confocal microscope (Nikon, Tokyo, Japan) equipped with a Nikon 10× planapochromate objective (numerical aperture, 0.75) and the corresponding filters.

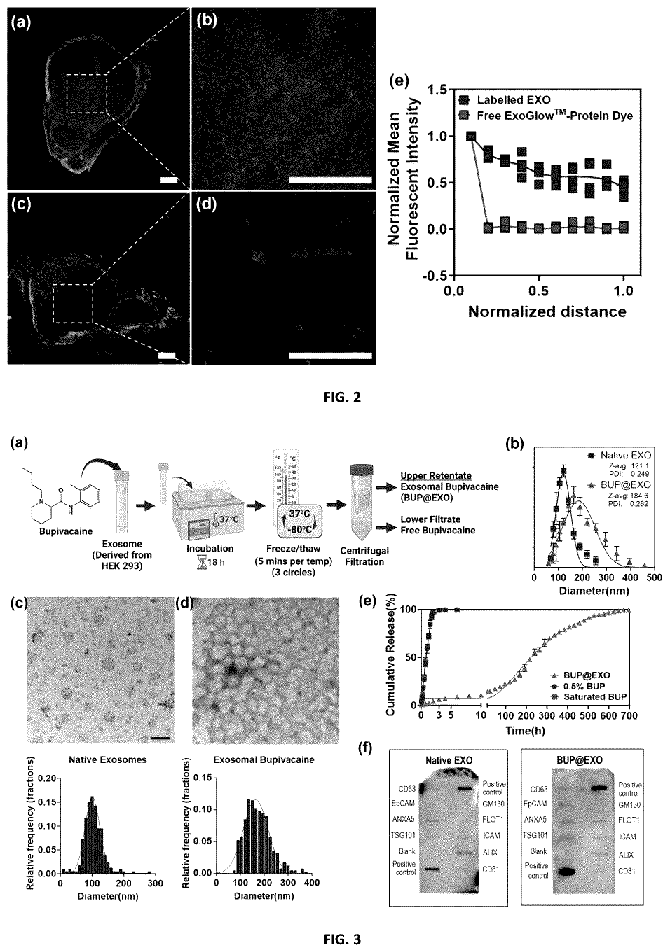

In the rats injected with ExoGlow™-Protein labeled exosomes, significant fluorescent intensity was detected in the interior portion of nerve section (FIG. 2a-b). In contrast, in the nerve of rats injected with the same dose of free ExoGlow™-Protein, the fluorescent signal was predominantly accumulated around the nerve fiber boundary, with no discernible fluorescence observed within the nerve section (FIG. 2c-d). Quantitative analysis revealed that ExoGlow™-Protein labeled exosomes penetrated deeply into the nerve of rats, with the fluorescence intensity only decreased slightly with increasing distance from the nerve fiber boundary (FIG. 2e). Notably, even at the core of the nerve section (where the normalized distance is 1 out of 1), the normalized mean fluorescent intensity reached an average value of 0.53 out of 1. In contrast, in the nerve of rats injected with free ExoGlow™-Protein, the normalized mean fluorescent intensity dropped sharply to less than 0.05 in regions with a normalized distance >0.1. These results provided compelling evidence of the exosomes' ability to cross the perineurium and enter nerves.

Following euthanasia, the sciatic nerve and adjacent muscle tissues were meticulously extracted. Muscle specimens underwent processing to yield slides stained with hematoxylin and eosin. A macroscopic dissection score was assigned based on the following criteria: 0=tissue planes obvious and easily separated; 1=tissue planes obvious but separated with some difficulty; 2=tissue planes adherent and separated with some difficulty; 3=tissue planes completely obliterated, could not separate surrounding tissues from nerve without cutting through them. Muscle samples underwent evaluation for inflammation (on a scale of 0-4) and myotoxicity (on a scale of 0-6). The inflammation score was a subjective measure of its severity. The myotoxicity score was based on two hallmark features of local anesthetic-induced myotoxicity: nuclear internalization and regeneration. Nuclear internalization is typified by myocytes that appear normal in size and chromicity but possess nuclei displaced from their typical peripheral location. Regeneration manifests as contracted myocytes with basophilic cytoplasm. The scoring was as follows: 0=normal; 1=perifascicular internalization; 2=deep internalization (>5 cell layers), 3=perifascicular regeneration, 4=deep regeneration, 5=hemifascicular regeneration, 6=holofascicular regeneration.

For nerve injury histology, samples underwent Epon-embedding and fixation using Karnovsky's KII Solution, which comprises 2.5% glutaraldehyde, 2.0% paraformaldehyde, and 0.025% calcium chloride in a 0.1 M sodium cacodylate buffer with a pH of 7.4. Subsequently, samples were post-fixed using osmium tetroxide, stained with uranyl acetate, and then dehydrated through a series of ethanol solutions. This was followed by infiltration with mixtures of propylene oxide and Epon. Sections, with a thickness of 0.5 μm, were then stained using toluidine blue, facilitating high-resolution light microscopy examination. Notably, neuropathological alterations were negligible across all injection groups, rendering a quantitative neurotoxicity analysis unnecessary.

In another experiment, rats were euthanized at the 48-hour mark. Blood samples were meticulously collected through two methods: a cardiac puncture, which involves drawing blood directly from the heart, and a separate collection from the femoral artery. Once collected, the blood samples were subjected to centrifugation, a process that separates the serum from other blood components. The isolated serum was then analyzed for creatine kinase activity using a colorimetric assay kit for creatine kinase activity. The intensity was quantified using a plate-reader, an instrument that measures absorbance levels.

In the rats injected with ExoGlow™-Protein labeled exosomes, significant fluorescent intensity was detected in the interior portion of nerve section (FIG. 2a-b). In contrast, in the nerve of rats injected with the same dose of free ExoGlow™-Protein, the fluorescent signal was predominantly accumulated around the nerve fiber boundary, with no discernible fluorescence observed within the nerve section (FIG. 2c-d). Quantitative analysis revealed that ExoGlow™-Protein labeled exosomes penetrated deeply into the nerve of rats, with the fluorescence intensity only decreased slightly with increasing distance from the nerve fiber boundary (FIG. 2e). Notably, even at the core of the nerve section (where the normalized distance is 1 out of 1), the normalized mean fluorescent intensity reached an average value of 0.53 out of 1. In contrast, in the nerve of rats injected with free ExoGlow™-Protein, the normalized mean fluorescent intensity dropped sharply to less than 0.05 in regions with a normalized distance >0.1. These results provided compelling evidence of the exosomes' ability to cross the perineurium and enter nerves.

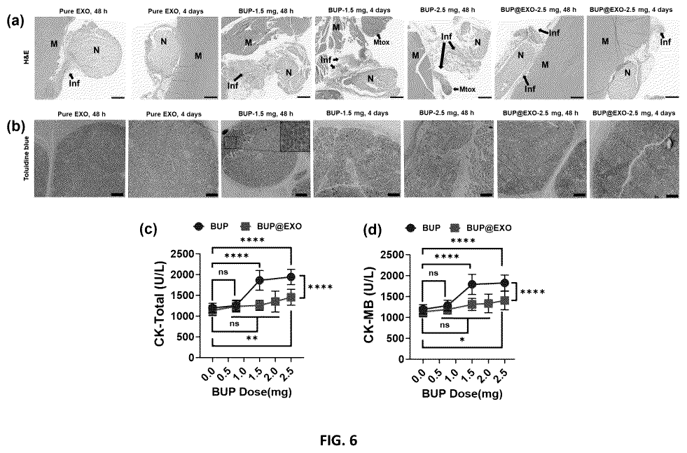

Rats injected with 0.3 mL BUP@EXO and free BUP formulations were euthanized 48 hours and 4 days after injection (n=4 at each respective time point). The sciatic nerve and surrounding tissues were harvested, sectioned, and stained for histologic assessment. Muscle tissues were processed for hematoxylin-eosin (H&E) staining. The myotoxicity and inflammation were quantified using a previously reported scoring system (Table 1, Table 2). When the rats were injected with free BUP, at the dosage of 0.75 mg, microscopic examination did not reveal myotoxicity or severe inflammation in rats at both 48-hour and 4-day intervals (inflammation scores range from 0 to 2, myotoxicity scores range from 0 to 1, FIG. 6a, Table 1, Table 2). When the dosage was elevated to 1.5 mg, the myotoxicity and the inflammation still remained in acceptable range at 48 hours (inflammation scores range from 0 to 2, myotoxicity scores range from 0 to 1, FIG. 6a, Table 1). However, at 4 days, 1 of 4 rats administered with 1.5 mg free BUP notably presented a myotoxicity with a score of 2 (FIG. 6a, Table 2). Furthermore, at the higher dosage of 2.5 mg, the severe inflammation and myotoxicity were observed at 48 hours, with myotoxicity scores up to 3 out of 6, and inflammation scores up to 3 out of 4. In comparison, when the rats were injected with BUP@EXO, at all the doses range from 0.75 mg to 2.5 mg, both the inflammation and myotoxicity were controlled at minimum (inflammation scores range from 0 to 1, myotoxicity scores range from 0 to 1.

Epon-embedded sections of the sciatic nerve were stained with toluidine blue. When the rats were injected with free BUP, 1 out of 4 tissues exposed to 1.5 mg of free BUP exhibited signs of neurotoxicity at the interval of 48 hours, notably in the peripheral regions of the nerve sections where solid nerve bundles appeared, an early manifestation of nerve bundle damage (indicated in zoom-in section of FIG. 6b). Furthermore, when 2.5 mg free BUP was injected, 3 out of 4 tissues presented significant nerve degeneration, which is determined by the density decrease of nerve fibers within the nerve section (FIG. 6b). In a stark contrast, the BUP@EXO formulations with doses range from 0.75 mg to 2.5 mg were found not to cause nerve damage at both 48-hour and 4-day intervals (FIG. 6b).

The favorable tissue response to BUP@EXO is attributed to the targeted delivery of BUP to nerves and sustained release of BUP by exosomes. Crucially, encapsulating BUP in exosomes resulted in reduced exposure of BUP to local tissues, thereby diminishing tissue reactions associated with BUP. On the other hand, the sustained release of BUP allows a low BUP concentration acting on nerve, avoiding nerve damage caused by the BUP overdose. This finding highlights the advantage of exosomal delivery in PNB penetration, effectively reducing the absorption of BUP by adjacent tissue.

It is important to recognize that the side effects of BUP and other local anesthetics go beyond just localized tissue reactions. A major concern with administering BUP is its potential harmful cardiotoxic effects. This can be particularly severe in certain instances, such as arrhythmias and cardiac asystole. Cardiotoxicity can be observed through increased activity of Creatine Kinase(CK), an family of enzymes that is released into the blood in response to lesions in contractile fibers, serve as biochemical markers for muscle damage, including myocardial injury. Elevated activity levels of CK, especially its cardiomyocyte isoform—Creatine Kinase-Myocardial Band (CK-MB), can be a warning sign for serious outcomes such as cardiac arrhythmias, heart failure, and other cardiovascular complications.

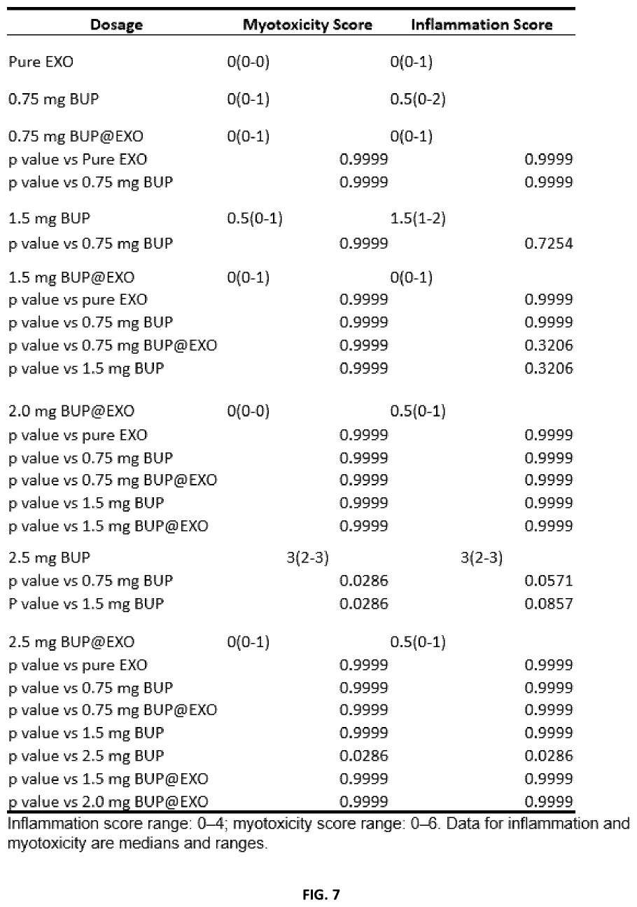

We conducted a comprehensive series of assessments to evaluate the potential cardiotoxicity caused by the sciatic injection of BUP@EXO by measuring the CK activity profile in rats. 48 hours after the injection with 0.3 mL of 1×PBS solutions containing free BUP or BUP@EXO, the rats were euthanized for blood sample collection. Two types of samples were obtained: from the femoral artery and cardiac puncture. These samples were analyzed to evaluate the activity in serum of total CK (CK-total) and its cardiomyocyte isoform (CK-MB). Results demonstrated that the administration of 0.75 mg free BUP did not result in significant alteration in both Creatine Kinase activity profiles. However, the injection of both 1.5 mg and 2.5 mg free BUP induced a considerable elevation in CK activity (FIG. 7b, 7c; p<0.0001 vs blank control). In comparison, compared to our blank control, the majority of administered BUP@EXO doses (0.75-2.0 mg equivalent bupivacaine) exhibited no significant alteration in the CK-profile, the only significant increase was observed at the BUP@EXO containing 2.5 mg BUP (FIG. 7b, 7c; p<0.01 vs blank control). Importantly, despite the observed alteration in CK activity, the administration of 2.5 mg BUP@EXO demonstrated a significant reduced elevation in CK activity compared with the same dose of free BUP (p<0.0001). These observed effects in enzymatic levels are particularly encouraging, indicating that exosomal encapsulation of BUP not only reduces the absorption of BUP by adjacent tissue, but also mitigates drug entry into the bloodstream. The reduced risk of cardiotoxicity, a notable concern associated with the clinical use of BUP, makes BUP@EXO a highly desirable alternative nerve-blocking agent to BUP for treating post-operative and chronic pain.

Previous reports have indicated that less than 1% of the perineurally injected local anesthetic can penetrate the perineurium and act on the peripheral nerve axons[1-3]. Co-administration of local anesthetics with chemical permeation enhancers (CPEs) has proven to be the most effective method for increasing the bioavailability of local anesthetics in the peripheral nerves and enhancing their analgesic effect. However, CPEs are generally corrosive, and excessive amounts can cause nerve damage. This study demonstrated that peripherally injected exosomes can cross the PNBs and deliver the loaded local anesthetic directly to the axons. This approach supports improved permeability and retention of the local anesthetic in the peripheral nerves without the side effects associated with CPEs.

The BUP@EXO formulation exhibits higher potency in peripheral nerve block and lower local tissue and systemic toxicity than not only pure bupivacaine but also Exparel. Exparel™ is a clinically used liposomal BUP formulation. Unlike the BUP@EXO formulation, which combines the function of BUP sustained release and the function of enhanced penetration to PNBs, Exparel only has the function of sustained release of BUP but does not have the function of crossing PNBs. In the same rat sciatic nerve model, a single injection of Exparel containing 7.86 mg of BUP induced a 4-hour sensory nerve blockade and was associated with obvious local myotoxicity and neurotoxicity. This is in significant contrast to the single injection of BUP@EXO, which demonstrated superior blockade duration at a significantly lower dosage. BUP@EXO produced a sensory nerve blockade lasting up to 12 hours with just 2.5 mg equivalent of BUP. This difference demonstrates the importance of the exosomes' inherent ability to penetrate the PNBs in enhancing nerve block efficacy and reducing side effects. The longer duration of action and lower risk of side effects compared to pure BUP and Exparel make the BUP@EXO formulation a clinically promising drug for acute post-operative and chronic pain management.

Exosomes can be used as a carrier for the promising nerve blocking agent, tetrodotoxin (TTX), known for its interference with the transmission of pain signals by blocking voltage-gated sodium channels. Compared to clinically used local anesthetics, TTX exhibits much higher potency in peripheral nerve blocking and carries no local tissue and myocardial toxicity. However, TTX is an extremely potent toxin, and an overdose can lead to TTX-related side effects, and even death. Despite clinical attempts to use TTX for pain treatment, the toxicity problem associated with TTX remains unresolved, and the clinical application of TTX has been hindered. The use of exosomes, which could mitigate the risk of TTX toxicity and enhance its efficacy in peripheral nerve block, similar to the approach with BUP, would bring substantial benefits to advancing the clinical practice of TTX in pain relief applications. TTX is a hydrophilic molecule, and it can be loaded into exosomes through an osmotic shock method, which is acclaimed as the most optimized technique for the encapsulation of hydrophilic molecules within exosomes. The method leverages a concentration gradient and osmotic pressure to encapsulate hydrophilic drugs. It involves initially subjecting exosomes to hypotonic conditions, inducing osmotic shock by exposing them to a high-osmotic-pressure environment containing the hydrophilic drug, followed by an osmotic adjustment to return the exosomes to regular osmotic pressure. Employing this method, the encapsulation efficiency achieved with pentoxifylline—a representative hydrophilic drug—reached an impressive 0.85 μg of drug per μg of protein-equivalent exosomes.

### Additional Embodiments

1. A composition comprising an anesthetic encapsulated in an exosome.
2. The composition according to a preceding embodiment, wherein the anesthetic comprises a local anesthetic, a sodium channel blocker, a peptide neurotoxin, a TRPV1 antagonist, a TRPV1 agonist, a COX inhibitor (either COX-1 or COX-2), NMDA antagonist, a GABA agonist, a barbiturate, or a combination thereof.
3. The composition according to a preceding embodiment, wherein the anesthetic comprises a local anesthetic.
4. The composition according to a preceding embodiment, wherein the anesthetic comprises a local anesthetic selected from acetamidoeugenol, alfadolone acetate, alfaxalone, amucaine, amolanone, amylocaine, articaine, benoxinate, benzocaine, betoxycaine, biphenamine, bupivacaine, burethamine, butacaine, butaben, butanilicaine, buthalital, butoxycaine, carticaine, centbucridine, 2-chloroprocaine, cocaethylene, cyclomethycaine cocaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutyl p-aminobenzoate, ketamine, leucinocaine mesylate, levoxadrol, lidocaine, lignocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, sameridine, tetracaine, thialbarbital, thimylal, thiobutabarbital, thiopental, tolycaine, trimecaine, and zolamine, a pharmaceutically acceptable salt thereof, and combinations thereof.
5. The composition according to a preceding embodiment, wherein the anesthetic comprises a local anesthetic selected from dibucaine, lidocaine, bupivacaine, benzocaine, tetracaine, prilocaine, pharmaceutically acceptable salts thereof, and combinations thereof.
6. The composition according to a preceding embodiment, wherein the local anesthetic comprises bupivacaine hydrochloride.
7. The composition according to a preceding embodiment, wherein the anesthetic comprises a TRPV1 antagonist, a TRPV1 agonist, or a combination thereof.
8. The composition according to a preceding embodiment, wherein the anesthetic comprises a TPRV1 agonist selected from N-vanillyl-alkanedienamides, N-vanillyl-alkanedienyls, N-vanillyl-cis-monounsaturated alkenamides, gingerol, shogaol, zingerone, eugenol, N-arachidonoyldopamine, olvanil, palvanil, resiniferatoxin, capsaicin, dihydrocapsaicin, norhydrocapsaicin, nordihydrocapsaicin, homocapsaicin, and homodihydrocapsaicin, pharmaceutically acceptable salts thereof and combinations thereof.
9. The composition according to a preceding embodiment, wherein the anesthetic comprises a TPRV1 antagonist selected from capsazepine, ruthenium red, pharmaceutically acceptable salts thereof, and combinations thereof.
10. The composition according to a preceding embodiment, wherein the anesthetic comprises a site 1 calcium channel blocker.
11. The composition according to a preceding embodiment, wherein the anesthetic comprises a site 1 calcium channel blocker selected from tetrodotoxin, batrachotoxin, saxitoxin, gonyautoxin, neosaxitoxin, decarbamoylsaxitoxin, pharmaceutically acceptable salts thereof, and combinations thereof.
12. The composition according to a preceding embodiment, wherein the anesthetic comprises a peptide neurotoxin.
13. The composition according to a preceding embodiment, wherein the anesthetic comprises a peptide neurotoxin selected from agatoxin, conotoxin, calciseptine, calcicludine, kurtoxin, cysteine-rich secretory proteins (CRISPs), glycerotoxin, phonetoxin, filistatoxin, SNX-482, protoxins, grammotoxin, pharmaceutically acceptable salts thereof, and combinations thereof.
14. The composition according to a preceding embodiment, wherein the anesthetic comprises a protein neurotoxin.
15. The composition according to a preceding embodiment, wherein the anesthetic comprises a protein neurotoxin comprising botulinum neuortoxin.
16. The composition according to a preceding embodiment, comprising a local anesthetic in an amount (in free base equivalents) from 0.1-100 mg, 0.1-50 mg, from 0.1-25 mg, from 0.1-10 mg, from 0.1-5 mg, from 0.1-2.5 mg, from 0.1-1 mg, from 0.5-5 mg, from 1-5 mg, from 2.5-5 mg, from 5-10 mg, from 10-25 mg, or from 25-50 mg.
17. The composition according to a preceding embodiment, comprising bupivacaine, or pharmaceutically acceptable salt thereof, in an amount (in free base equivalents) from 0.1-50 mg, from 0.1-25 mg, from 0.1-10 mg, from 0.1-5 mg, from 0.1-2.5 mg, from 0.1-1 mg, from 0.5-5 mg, from 1-5 mg, from 2.5-5 mg, from 5-10 mg, from 10-25 mg, or from 25-50 mg.
18. The composition according to a preceding embodiment, comprising site 1 calcium channel blocker in an amount (in free base equivalents) from 0.1-50 μg, from 0.1-25 μg, from 0.1-10 μg, from 0.1-5 μg, from 0.1-2.5 μg, from 0.1-1 μg, from 0.5-5 μg, from 1-5 μg, from 2.5-5 μg, from 5-10 μg, from 10-25 μg, or from 25-50 μg. 19. The composition according to a preceding embodiment, wherein the exosome is derived from an animal cell, a plant cell, a fungal cell, or a bacterial cell.
20. The composition according to a preceding embodiment, wherein the exosome is derived from a mammalian cell.
21. The composition according to a preceding embodiment, wherein the exosome is derived from a mammalian stem cell.
22. The composition according to a preceding embodiment, wherein the exosome is derived from a mammalian embryonic stem cell.
23. The composition according to a preceding embodiment, wherein the exosome is derived from a human embryonic kidney cell.
24. The composition according to a preceding embodiment, wherein the exosome is derived from a human embryonic kidney 293 cell.
25. The composition according to a preceding embodiment, wherein the exosome has an average particle diameter (measured by dynamic light scattering) from 50-1000 nm, from 100-1000 nm, from 250-1000 nm, from 500-1000 nm, from 50-500 nm, from 100-500 nm, from 150-500 nm, from 250-500 nm, from 50-250 nm, from 100-250 nm, from 150-250 nm, from 200-250 nm, from 100-150 nm, or from 150-200 nm.
26. The composition according to a preceding embodiment, further comprising at least one pharmaceutically acceptable excipient.
27. The composition according to a preceding embodiment, further comprising sterile water, PBS, HEPES, HEPPS, HEPBS, PIPES, TAPS, Tris, TAPSO, MOPS, MES, cacodylate, TES, tricine, or a combination thereof.
28. The composition according to a preceding embodiment, further comprising a preservative.
29. The composition according to a preceding embodiment, further comprising a preservative comprising benzyl alcohol, benzalkonium chloride, butyl paraben, chlorobutanol, meta cresol, chlorocresol, methyl paraben, phenyl ethyl alcohol, propyl paraben, phenol, benzonic acid, sorbic acid, sodium benzonate, bronidol, propylene glycol, or a combination thereof.
30. The composition according to a preceding embodiment, further comprising a surfactant.
31. The composition according to a preceding embodiment, further comprising a surfactant comprising tocopheryl polyethylene glycol succinate, polyethylene glycol, Triton X-100, Tween 80, poloxamers, polysorbates, polidocanol, pentaethylene glycol monododecyl ether, lauryl glucoside, maltoside, monolaurin, mycosubtilin, narrow-range ethoxylate, nonidet P-40, nonoxynols, NP-40, octaethylene glycol monododecyl ether, N-octyl beta-D-thioglucopyranoside, octyl glucoside, oleyl alcohol, alkyl polyglycoside, cetomacrogol, cetostearyl clcohol, cetyl alcohol, cocamides, decyl glucoside, decyl polyglucose, glycerol monostearate, IGEPAL, isoceteth-20, or a combination thereof.
32. The composition according to a preceding embodiment, further comprising an antioxidant.
33. The composition according to a preceding embodiment, further comprising an antioxidant comprising vitamin A, vitamin A derivative, vitamin C, vitamin C derivatives, vitamin E, vitamin E derivatives, polyphenols, beta-carotene, lutein, lycopene, selenium, or a combination thereof.
34. The composition according to a preceding embodiment, wherein the composition is an injectable composition or a topically applied composition.
35. The composition according to a preceding embodiment, wherein the composition is a topically applied composition comprising a pharmaceutically acceptable vehicle.
36. The composition according to a preceding embodiment, wherein the exosome loses 10% or less of the anesthetic over a period of two weeks when stored at 4 C, and loses 20% or more of the 26anesthetic over a period of 48 hours when stored at 23° C.
37. A method of making the composition according to a preceding embodiment, comprising contacting an exosome with a solution comprising the anesthetic.
38. The method according to a preceding embodiment, wherein the solution has a temperature from 23-40° C., from 23-37° C., from 30-37° C., or from 33-37° C.
39. The method according to a preceding embodiment, wherein the solution is a buffered aqueous solution.
40. The method according to a preceding embodiment, wherein the solution is phosphate buffered saline.
41. The method according to a preceding embodiment, wherein the solution is 1× phosphate buffered saline.
42. The method according to a preceding embodiment, wherein the concentration of the anesthetic in the solution is from 1-250 mg/ml, from 1-100 mg/ml, from 1-75 mg/ml, from 1-50 mg/ml, from 1-25 mg/ml, from 1-10 mg/ml, from 5-15 mg/ml, from 10-20 mg/ml, from 10-50 mg/ml, from 25-75 mg/ml, or from 50-100 mg/ml.
43. The method according to a preceding embodiment, wherein the following the contacting of the exosome with the solution comprising the anesthetic, the exosome is subjected to one or more freeze-thaw cycles.
44. The method according to a preceding embodiment, wherein the freeze-thaw cycle comprises a freeze period at a temperature from −120° C. to −50° C., followed by a thaw period at a temperature from 10-40° C.
45. The method according to a preceding embodiment, wherein the freeze-thaw cycle comprises a freeze period at a temperature from −90° C. to −70° C., preferably about −80° C., followed by a thaw period at a temperature from 23-40° C., preferably about 37° C.
46. The method according to a preceding embodiment, comprising at least 3 freeze-thaw cycles.
47. The method according to a preceding embodiment, wherein the freeze period is for a duration from 2-30 minutes, from 2-20 minutes, from 2-15 minutes, from 2-10 minutes, from 2-5 minutes, from 4-10 minutes, from 8-15 minutes, or from 10-30 minutes.
48. The method according to a preceding embodiment, wherein the thaw period is for a duration from 2-30 minutes, from 2-20 minutes, from 2-15 minutes, from 2-10 minutes, from 2-5 minutes, from 4-10 minutes, from 8-15 minutes, or from 10-30 minutes.
49. The method according to a preceding embodiment, wherein the exosomes are first contacted with a solution of pure water, then contacted with a solution comprising a therapeutic agent, and then contacted with a solution comprising a buffer, to give a final composition having an osmolality from 200-400 mOsmol/kg.
50. A method for producing anesthesia in a patient in need thereof, comprising administering to the patient the composition according to a preceding embodiment.
51. The method according to a preceding embodiment, wherein the composition is administered by injection.
52. The method according to a preceding embodiment, wherein the composition is administered by injection to a location adjacent to a nerve.
53. The method according to a preceding embodiment, wherein the composition is administered by injection to a location adjacent to a peripheral nerve.
54. The method according to a preceding embodiment, wherein the composition is administered by injection to a location adjacent to the celiac plexus nerve, the epidural nerve, the genicular nerve, the intercostal nerve, the lumbar sympathetic nerve, the occipital nerve, the pudendal nerve, the stellate ganglion nerve, or the trigeminal nerve.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches

What is claimed is:

1. A composition comprising an anesthetic encapsulated in an exosome.

2. The composition according claim 1, wherein the anesthetic comprises a local anesthetic, a sodium channel blocker, a peptide neurotoxin, a TRPV1 antagonist, a TRPV1 agonist, a COX inhibitor (either COX-1 or COX-2), NMDA antagonist, a GABA agonist, a barbiturate, or a combination thereof.

3. The composition according to claim 1, wherein the anesthetic comprises a local anesthetic.

4. The composition according to claim 1, wherein the anesthetic comprises bupivacaine hydrochloride.

5. The composition according to claim 1, wherein the anesthetic comprises a site 1 calcium channel blocker.

6. The composition according to claim 1, wherein the anesthetic tetrodotoxin.

7. The composition according to claim 1, comprising a local anesthetic in an amount (in free base equivalents) from 0.1-100 mg.

8. The composition according to claim 1, wherein the exosome is derived from a mammalian embryonic stem cell.

9. The composition according to claim 1, wherein the exosome has an average particle diameter (measured by dynamic light scattering) from 50-1000 nm.

10. The composition according claim 1, further comprising sterile water, PBS, HEPES, HEPPS, HEPBS, PIPES, TAPS, Tris, TAPSO, MOPS, MES, cacodylate, TES, tricine, or a combination thereof.

11. The composition according to claim 1, wherein the exosome loses 10% or less of the anesthetic over a period of two weeks when stored at 4 C, and loses 20% or more of the anesthetic over a period of 48 hours when stored at 23° C.

12. A method of making the composition according to claim 1, comprising contacting an exosome with a solution comprising the anesthetic.

13. The method according to claim 12, wherein the concentration of the anesthetic in the solution is from 1-250 mg/ml.

14. The method according to claim 12, wherein after the exosome is contacted with the solution comprising the anesthetic the exosome is subjected to one or more freeze-thaw cycles.

15. The method according to claim 14, wherein the freeze-thaw cycle comprises a freeze period at a temperature from −120° C. to −50° C., followed by a thaw period at a temperature from 10-40° C.

16. The method according to claim 14, comprising at least 3 freeze-thaw cycles.

17. The method according to claim 12, wherein the exosomes are first contacted with a solution of pure water, then contacted with a solution comprising a therapeutic agent, and then contacted with a solution comprising a buffer, to give a final composition having an osmolality from 200-400 mOsmol/kg.

18. A composition comprising an anesthetic encapsulated in an exosome, produced according to the method of claim 12.

19. A method for producing anesthesia in a patient in need thereof, comprising administering to the patient the composition according to claim 1.

20. The method according to claim 19, wherein the composition is administered by injection.
